(19) United States
(12) Patent Application Publication
(10) Pub. No.: US 2007/0032491 A1
(43) Pub. Date: Feb. 8, 2007

(54) Combination of a glycine/nmda antagonist and a tachykinin nk-1 receptor antagonist for use in the treatment of neurodegeneration

(76) Inventor: Jose Luis Castro Pineiro, Bishops Stortford (GB)

Correspondence Address: MERCK AND CO., INC, P O BOX 2000, RAHWAY, NJ, 07065-0907, US

(21) Appl. No.: 10/555,711

(22) PCT Filed: May 4, 2004
(86) PCT No.: PCT/GB04/01926

§ 371 Date: Nov. 2, 2005

(30) Foreign Application Priority Data
May 12, 2003 (GB) 0310881.8

(51) Int. Cl. A61K 31/5377; A61K 31/498

(52) U.S. Cl. 514/235.5; 514/249

(57) ABSTRACT

The present invention relates to a pharmaceutical formulation comprising a compound which is active as an antagonist of the strychnine-insensitive glycine modulatory site of the N-methyl-D-asparate (NMDA) receptor in combination with a tachykinin NK-1 receptor antagonist, for use in the treatment of neurodegeneration arising, in particular, from stroke or cerebral ischemia.

The present invention relates to a pharmaceutical composition comprising a combination of active ingredients. More particularly, the invention concerns a pharmaceutical formulation comprising a compound which is active as an antagonist of the strychnine-insensitive glycine modulatory site of the N-methyl-D-asparate (NMDA) receptor (hereinafter referred to as a “glycine NMDA antagonist”) in combination with a tachykinin NK-1 receptor antagonist, for use in the treatment of neurodegeneration arising, in particular, from stroke or cerebral ischemia.

Glycine/NMDA antagonists are well known from the art to be of benefit in the treatment of acute neurodegenerative disorders arising from events such as stroke, transient ischemic attack, peri-operative ischemia, global ischemia (following cardiac arrest), and traumatic head injury to the brain or spinal cord. In addition, glycine/NMDA antagonists may be of use in treating certain chronic neurological disorders such as senile dementia, Parkinson's disease and Alzheimer's disease. They may also have utility in conditions in which peripheral nerve function has been impaired, such as retinal and macular degeneration.

Glycine/NMDA antagonists have, moreover, been reported as being beneficial in treating epilepsy; anxiety; substance abuse and/or addiction, e.g. alcoholism; pain; hearing disorders, e.g. tinnitus; migraine; and psychiatric disorders such as schizophrenia. However, mechanism-based side effects, principally including nausea and vomiting, have been reported following administration of certain glycine NMDA antagonists during clinical trials.

The neuropeptide receptors for substance P (SP; neurokinin-1; NK-1) are widely distributed throughout the mammalian nervous system (especially the brain and spinal ganglia), circulatory system and peripheral tissues (especially the duodenum and jejunum), and are involved in regulating a variety of diverse biological processes. These include the sensory perception of olfaction, vision, audition and pain; movement control; gastric motility; vasodilation; salivation; and micturition.

Substance P is a naturally occurring undecapeptide belonging to the tachykinin family of peptides, the latter being so-named because of their prompt contractile action on extravascular smooth muscle tissue. In addition to SP, the known mammalian tachykinins include neurokinin A and neurokinin B. The current nomenclature designates the receptors for substance P, neurokinin A and neurokinin B as neurokinin-1, neurokinin-2 and neurokinin-3 respectively.

Tachykinin neurokinin-1 (NK-1; substance P) receptor antagonists are being developed for the treatment of a number of physiological disorders associated with an excess or imbalance of tachykinins, in particular SP. Examples of such conditions include disorders of the central nervous system including anxiety, depression and psychosis. Recently, the tachykin NK-1 receptor antagonist aprepitant [2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine] has been approved by the US Food and Drug Administration (FDA) for use in preventing the acute and delayed nausea and vomiting associated with cancer chemotherapeutic agents, including high-dose cisplatin.

It has now been found that the co-administration of a glycine/NMDA antagonist in conjunction with a tachykinin NK-1 receptor antagonist provides beneficial results in the treatment of neurodegeneration arising, in particular, from stroke or cerebral ischemia.

The present invention accordingly provides a method for the treatment of neurodegeneration which comprises administering to a patient in need of such treatment, either simultaneously, separately or sequentially, a combination of a glycine/NMDA antagonist and a tachykinin NK-1 receptor antagonist.

The present invention also provides the use of a combination of a glycine/NMDA antagonist and a tachykinin NK-1 receptor antagonist for the manufacture of a medicament for the treatment of neurodegeneration.

In another aspect, the present invention provides a pharmaceutical composition comprising a glycine/NMDA antagonist and a tachykinin NK-1 receptor antagonist in association with a pharmaceutically acceptable carrier.

In a further aspect, the present invention provides a product containing a glycine/NMDA antagonist and a tachykinin NK-1 receptor antagonist as a combined preparation for simultaneous, separate or sequential use in the treatment of neurodegeneration.

In the normal practice of the invention, the glycine NMDA antagonist and the tachykinin NK-1 receptor antagonist will usually be administered to a patient within a reasonable period of time, which will typically be up to about one hour apart. The compounds may be in the same pharmaceutical carrier and therefore administered simultaneously. They may be in separate pharmaceutical carriers and administered simultaneously, by mixing the materials just prior to administration. They may alternatively be in different dosage forms which can be taken simultaneously, or administered sequentially.

Typical glycine/NMDA antagonists of use in the present invention are, for example, described in EP-A-0481676. Preferred glycine/NMDA antagonists of use in this invention include UK-240,455 and UK-333,747, disclosed in WO 96/09295 [Example 80(d)] and WO 98/38186 (derived from WO 97/32873) respectively, the chemical structures of which are as follows:

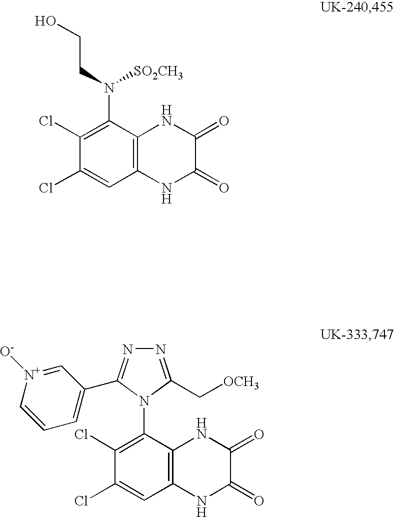

The tachykinin NK-1 receptor antagonists of use in the present invention may be peptidal or non-peptidal in nature. However, the use of a non-peptidal tachykinin NK-1 receptor antagonist is preferred. In a preferred embodiment, the tachykinin NK-1 receptor antagonist is a CNS-penetrant tachykinin NK-1 receptor antagonist. In addition, for convenience the use of an orally active tachykinin NK-1 receptor antagonist is preferred. To facilitate dosing, it is also preferred that the tachykinin NK-1 receptor antagonist is a long acting tachykinin NK-1 receptor antagonist. An especially preferred class of tachykinin NK-1 receptor antagonists of use in the present invention comprises those compounds which are both orally active and long acting.

Tachykinin NK-1 receptor antagonists of use in the present invention are fully described, for example, in U.S. Pat. Nos. 5,162,339, 5,232,929, 5,242,930, 5,373,003, 5,387,595, 5,459,270, 5,494,926, 5,496,833 and 5,637,699; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942, 97/21702 and 97/49710; and British Patent Publication Nos. 2 266 529, 2 268 931, 2 269 170, 2 269 590, 2 271 774, 2 292 144, 2 293 168, 2 293 169 and 2 302 689.

A preferred tachykinin NK-1 receptor antagonist of use in the present invention is aprepitant (supra), disclosed in WO 95/16679.

In a preferred embodiment of the present invention, UK-240,455 or UK 333,747 may be co-administered, as described herein, with aprepitant.

The pharmaceutical composition according to the present invention may conveniently be adapted for administration orally, rectally or parenterally. For oral administration, the formulation may be presented in the form of tablets, pills, capsules, powders or granules; for parenteral administration, sterile parenteral solutions or suspensions may conveniently be utilised; and for rectal administration, the formulation may conveniently be in the form of suppositories. Suitably, the pharmaceutical compositions in accordance with the invention may be presented in the form of a kit of parts adapted for simultaneous, separate or sequential administration.

The compositions may be formulated by conventional methods well known in the pharmaceutical art, for example as described in Remington: The Science and Practice of Pharmacy, Mack Publishing Company, 19th Edition, 1995.

For administration in combination, the glycine/NMDA antagonist and the tachykinin NK-1 receptor antagonist may be presented in a ratio which is consistent with the manifestation of the desired effect. In particular, the molar ratio of the glycine/NMDA antagonist to the tachykinin NK-1 receptor antagonist will suitably be approximately 1 to 1. Preferably, this ratio will be between 0.001 to 1 and 1000 to 1, and especially from 0.01:1 to 100:1.

For co-administration with a tachykinin NK-1 receptor antagonist in the treatment of neurodegeneration, the glycine/NMDA antagonist may suitably be administered at a daily dosage of about 0.001 to 250 mg/kg, typically about 0.005 to 100 mg/kg, more particularly about 0.01 to 50 mg/kg, and especially about 0.05 to 10 mg/kg. For co-administration with a glycine/NMDA antagonist in the treatment of neurodegeneration, the tachykinin NK-1 receptor antagonist may suitably be administered at a daily dosage of about 0.001 to 250 mg/kg, typically about 0.005 to 100 mg/kg, more particularly about 0.01 to 50 mg/kg and especially about 0.05 to 10 mg/kg. The active ingredients will typically be co-administered on a regimen of 1 to 4 times per day.

The following non-limiting Examples serve to illustrate the present invention.

### EXAMPLES 1 TO 4

Tablet Preparation

Tablets containing UK-240,455 and aprepitant, or UK-333,747 and aprepitant, were prepared as follows:

| | Example 1 | Example 2 |
|---|---|---|
| UK-240,455 | 5.0 mg | 10.0 mg |
| Aprepitant | 10.0 mg | 10.0 mg |
| Microcrystalline cellulose | 42.0 mg | 39.5 mg |
| Modified food corn starch | 42.0 mg | 39.5 mg |
| Magnesium stearate | 1.0 mg | 1.0 mg |
| | Example 3 | Example 4 |
| UK-333,747 | 5.0 mg | 10.0 mg |
| Aprepitant | 10.0 mg | 10.0 mg |
| Microcrystalline cellulose | 42.0 mg | 39.5 mg |
| Modified food corn starch | 42.0 mg | 39.5 mg |
| Magnesium stearate | 1.0 mg | 1.0 mg |

All of the active ingredients, cellulose, and a portion of the corn starch are mixed and granulated to 10% corn starch paste. The resulting granulation is sieved, dried and blended with the remainder of the corn starch and magnesium stearate. The resulting granulation is then compressed into tablets.

1-8. (canceled)

9. A method for the treatment of neurodegeneration which comprises administering to a patient in need of such treatment a glycine NMDA antagonist and a tachykinin NK-1 receptor antagonist.

10. The method of claim 9 wherein the glycine/NMDA antagonist is:

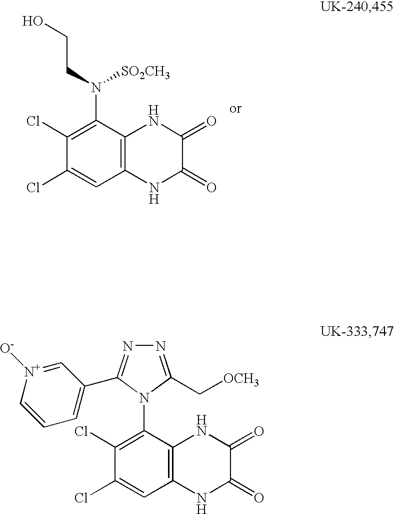

11. The method of claim 10 wherein the glycine/NMDA antagonist is UK-240,455.

12. The method of claim 10 wherein the glycine/NMDA antagonist is UK-333,747.

13. The method of claim 9 wherein the tachykinin NK-1 receptor antagonist is aprepitant.

14. The method of claim 9 wherein the neurodegeneration results from stroke.

15. The method of claim 9 wherein the neurodegeneration results from cerebral ischemia.

16. A pharmaceutical composition which comprises a glycine/NMDA antagonist, a tachykinin NK-1 receptor antagonist and a pharmaceutically acceptable carrier.
